(19) United States
(12) Patent Application Publication
(10) Pub. No.: US 2007/0073116 A1
(43) Pub. Date: Mar. 29, 2007

(54) Patient identification using physiological sensor

(76) Inventors: Massi E. Kiani, Laguna Niguel, CA (US); Yassir Abdul-Hafiz, Irvine, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/505,244

(22) Filed: Aug. 16, 2006

Related U.S. Application Data
(60) Provisional application No. 60/709,048, filed on Aug. 17, 2005.

(51) Int. Cl. A61B 5/00

(52) U.S. Cl. 600/310

(57) ABSTRACT

A patient information tracking system is disclosed that implements a physiological sensor system used to acquire information related to the wearer of a physiological sensor. The sensor system includes a physiological sensor adapted to be attached to a patient and includes at least one emitter and a photodetector. The sensor system further includes a positioning element to position the sensor such that the at least one emitter is sufficiently proximate the detector to acquire information from an identification element worn by the patient. A method for using a physiological sensor system to acquire information related to the wearer of a sensor is also provided. The sensor may also include a securing portion configured to couple to the sensor portion of the wearer. The sensor may also include a security wire and a memory device for retaining the wearer's information.

### REFERENCE TO RELATED APPLICATION

The present application claims priority benefit under 35 U.S.C. §119(e) from U.S. Provisional Application No. 60/709,048, filed Aug. 17, 2005, entitled “Patient Identification Using Physiological Sensor,” which is incorporated herein by reference.

### BACKGROUND OF THE INVENTION

1. Field of the Invention

The present invention relates in general to physiological sensors for patient monitoring.

2. Description of the Related Art

Recent years have seen a wide variety of physiological sensors being used for patient monitoring in caregiving facilities such as hospitals, nursing homes, and the like. One particular type of patient monitoring, pulse oximetry, is a widely accepted noninvasive procedure for measuring the oxygen saturation level of arterial blood, an indicator of the oxygen status of the blood. A pulse oximeter generally operates with one or more light emitting diodes (LEDs) that are placed on one side of a medium while a photodetector is placed on an opposite side of the medium. An artisan will also recognize other general operating paradigms, such as a reflective paradigm where the LEDs and photodetector are placed on the same side. In general, the foregoing pulse oximeters are used to measure a patient's blood oxygen saturation.

Conventional physiological sensors are disposable, reusable, or combinations of the two. A disposable sensor is generally attached to the patient with an adhesive wrap. A reusable sensor may be shaped roughly like a clip or clothespin that is easily attached and removed from, for example, a digit, earlobe, or the like. Combination sensors can include reusable circuitry that employs a disposable attachment mechanism, such as adhesive tape or bandage. Examples of each of the foregoing physiological sensors adapted for pulse oximetry are commercially available from Masimo Corporation of Irvine, Calif. Specific examples are U.S. Pat. Nos. 6,256,523 and 6,580,086, which are incorporated by reference herein.

During a patient's stay at a caregiver facility, such as a hospital, the patient may be moved to various rooms for tests, operations, or other procedures or may simply move themselves for activities, exercise, visitors, or the like. As patients move, it becomes increasingly difficult for caregivers to identify the patient. Hospital staff typically identifies patients by manually taking down the patient's information and then inputting that information into a computer. This procedure can be repetitious and time consuming, particularly in a time of emergency.

For these and other reasons, some caregivers have moved to identification bracelets to help identify patients, and in the case of newborns, the newborn's parents. While these bracelets or wristbands signify a significant advancement in patient identification, they still suffer from a variety of drawbacks. For example, many wristbands simply alphanumerically identify patients. Such wristband mechanisms still employ caregivers to manually record the alphanumeric information as the patient is moved. Other wristbands include encoded computer readable information such as bar code information. In at least one system, the caregiver facility uses modified pulse oximetry sensors to collect the barcode information in a more automated fashion. Such modified sensors include the drawback of employing specialty sensors that can be costly to implement. Based on the foregoing, significant and costly drawbacks exist in conventional oximetry sensors and patient information tracking.

Thus, a need exists for an oximetry sensor with the advantages of the disposable and reusable sensors combined with the ability to identify or recognize patients and retain patient information. To overcome some of the foregoing drawbacks, sensor designers have come up with a modified wristband and reusable-pulse-oximeter sensor combination.

### SUMMARY OF THE INVENTION

The present invention involves several different embodiments related to identifying a patient by a physiological sensor system. In one embodiment, a sensor is configured to identify a unique bar code that is placed on a patient's identification bracelet. Preferably, the sensor shines light onto the bar code, and the light is reflected back to the sensor. The sensor is able to identify the unique bar code corresponding to that patient, and hence, identifies the patient. In some embodiments, a positioning device may facilitate positioning of the sensor.

In another embodiment, the sensor may be connected to the patient's identification bracelet through in a variety of configurations and means. The sensor may be attached to the bracelet, for example, by adhesive, a clasp, a rivet, or the sensor may be integrally formed with the bracelet. In a further embodiment, the sensor may include a memory device that retains patient information. In this embodiment, when the sensor is connected to operating equipment and monitors, the patient identification information may be obtained from the memory device.

Various embodiments of the patient information tracking system disclosed herein also include a physiological sensor system usable to acquire information related to the wearer of a physiological sensor. The sensor system includes a physiological sensor that is adapted to be attached to a patient and includes at least one emitter and a photodetector. The system further includes a positioning element that positions the physiological sensor such that the at least one emitter is sufficiently proximate the detector to acquire information from an identification element worn by the patient.

In a further embodiment, a method of using a physiological sensor system to acquire information related to the wearer of a physiological sensor is provided. The method includes the steps of providing a physiological sensor including at least one emitter and a photodetector and providing a positioning element that positions the physiological sensor such that the emitter is sufficiently proximate the detector to acquire information from an identification element on the patient. The method further includes acquiring information from an identification element on the patient through the physiological sensor.

In yet another embodiment, a pulse oximetry sensor is provided. The pulse oximetry sensor includes a sensor portion having at least one emitter and a photodetector and a securing portion sized and configured to couple the sensor portion to a patient.

For purposes of summarizing the invention, certain embodiments, advantages, and novel features of the invention have been described herein. Of course, it is to be understood that not necessarily all such embodiments, advantages, or features are required in any particular embodiment of the invention.

### BRIEF DESCRIPTION OF THE DRAWINGS

FIG. 1A illustrates a side view of a pulse oximeter sensor with a positioning clip.

FIG. 1B illustrates a side view of the pulse oximeter sensor of FIG. 1A identifying a patient by reading a bar code.

FIG. 2 illustrates a side view of the pulse oximeter sensor with a guide, showing optical channels in broken lines to represent transparent or translucent channels.

FIG. 3 illustrates a side view of the pulse oximeter sensor with a positioning clip and guide combination.

FIG. 4 illustrates a side view of the pulse oximeter sensor with a fitted clamp.

FIG. 5 illustrates a perspective view of a pulse oximeter sensor identifying a patient by reading a bar code.

FIG. 6A illustrates a perspective view of the pulse oximeter sensor having a reusable portion, a disposable portion, and a securing portion extending from the disposable portion.

FIG. 6B illustrates a perspective view of the pulse oximeter sensor having a reusable portion, a disposable portion, and a securing portion extending from the reusable portion.

FIG. 7A illustrates a perspective view of the pulse oximeter sensor with the securing portion coupled to the identification bracelet.

FIG. 7B illustrates a perspective view of the pulse oximeter sensor with the securing portion integrally formed with the identification bracelet.

FIG. 7C illustrates a perspective view of the pulse oximeter sensor with the securing portion coupled to the identification bracelet via an identification bracelet clasp.

FIG. 8A illustrates a perspective view of the pulse oximeter sensor reading the bar code of an identification bracelet, a portion of the bracelet being transparent or translucent.

FIG. 8B illustrates a side view of a reusable pulse oximeter sensor reading the bar code of an identification bracelet, a portion of the bracelet being transparent or translucent.

### DETAILED DESCRIPTION OF THE PREFERRED EMBODIMENT

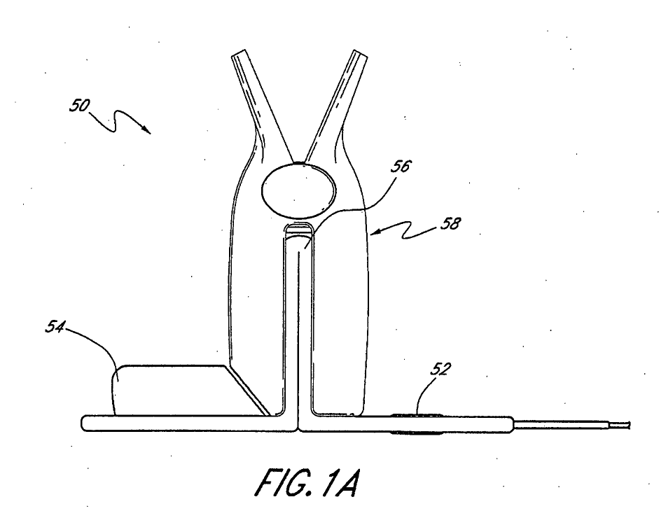

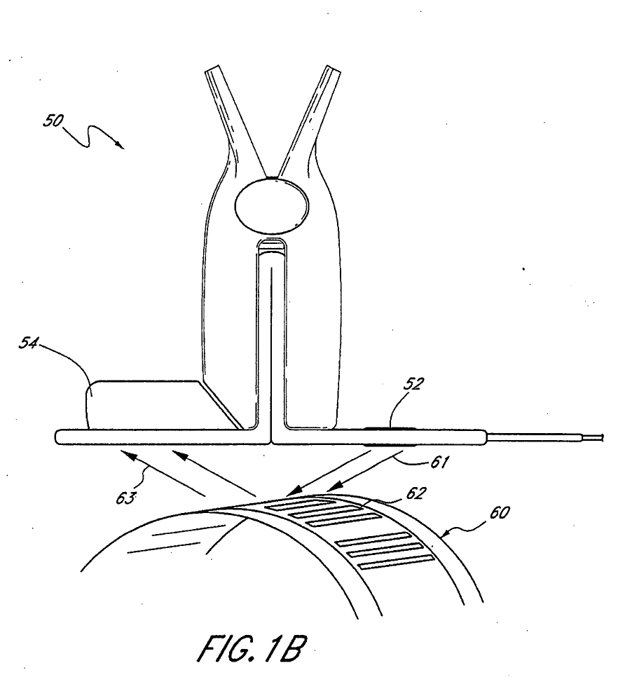

FIG. 1A illustrates one embodiment of a physiological sensor 50 configured to identify a patient. In this embodiment, the sensor 50 is preferably an oximetry sensor with an emitter 52 and a photodetector 54. The distance between the emitter 52 and the photodetector 54 is reduced by folding a portion 56 of the sensor between the emitter 52 and photodector 54, thereby creating sufficient proximity between the emitter 52 and the detector 54 that they can be employed in the acquisition of patient information from, for example, encoded information such as a bar code. Thus, in this embodiment, the existing electronic elements of the pulse oximeter sensor are advantageously positioned to perform bar code reading functionality. Such positioning can be accomplished through a variety of low cost structures or mechanisms, examples of which are described herein with reference to FIGS. 1A, 2, 3, and 4. However, an artisan will recognize from the disclosure herein other mechanisms for properly positioning the electronic elements of a pulse oximeter sensor.

As disclosed, an embodiment of the sensor 50 includes the folded portion 56 being held in place by a positioning clip 58. The clip 58 is preferably configured to facilitate gripping and releasing of the clip 58 by a user.

FIG. 1B illustrates the physiological sensor 50 of FIG. 1A identifying a patient. In one embodiment, a patient in a caregiver facility, such as a hospital, receives an identification bracelet 60. The identification bracelet 60 may include a transparent cover, under which may be placed a piece of paper that provides patient identification information. A bar code 62 may be provided on the identification bracelet 60 that uniquely identifies individual patients of the caregiver facility. An artisan will recognize from the disclosure herein that the bar code could be printed directly on plastic or the like.

The physiological sensor 50 of FIG. 1A is shown reading such the bar code 62 in FIG. 1B. Identification is performed by passing the sensor 50 over the bar code 62 at, for example, a relatively constant speed. The emitter directs light 61 from the emitter 52 to the identification bracelet 60. The emitted light 61 is reflected from the identification bracelet, and the reflected light 63 is detected by the photodetector 54. By identifying the relative space between the bar code 62 patterns, the sensor 50 is able to identify the unique pattern corresponding to the patient. In one embodiment, the signal is sent from the sensor 50 to an oximeter, and the oximeter identifies the bar code 62 corresponding to the patient. In another embodiment, the sensor 50 is configured to identify the patient by analyzing the bar code 62 pattern.

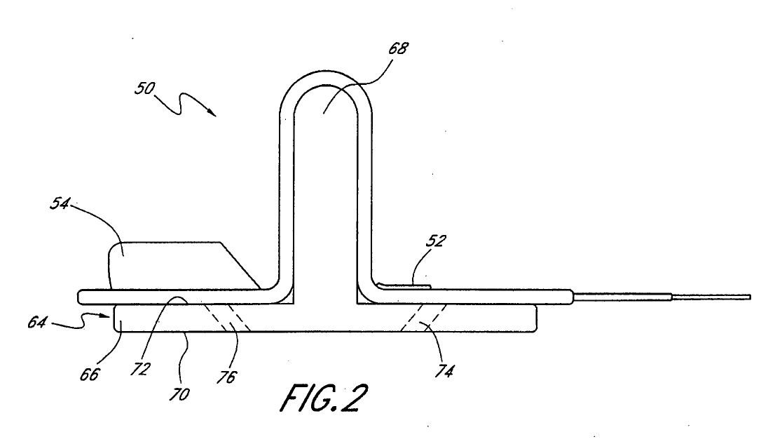

A further embodiment is illustrated in FIG. 2. In this embodiment, the sensor 50 is coupled to a guide 64. The guide 64 may comprise an application portion 66 and a gripping portion 68. In this embodiment, a caregiver conforms the sensor 50 to the shape of the gripping portion 68, thereby reducing the distance between the emitter 52 and the photodetector 54. The gripping portion 68 is preferably a sufficient length to reduce the distance between the emitter 52 and the photodetector 54 such that emission of light from the emitter 52 will be detected by the photodector 54. The application portion 66 of the guide 64 is preferably coupled to the gripping portion 68 and preferably comprises an application side 70 and a sensor side 72. The application side 70 faces the identification bracelet while the sensor side is adjacent to the sensor 50.

In one embodiment, the application portion 66 comprises a first channel 74 extending from the sensor side 72 to the application side 70, through which light may be directed from the emitter 52 to the identification bracelet. The application portion 66 also preferably comprises a second channel 76 adjacent the photodetector 54, such that light may be directed from the application side 70 to the sensor side 72 for detection by the photodetector 54. In another embodiment, the application portion 66 may not comprise channels, but may be transparent or translucent, thereby permitting passage of light to pass to and from the identification bracelet. In yet another embodiment, some or all of the guide 64 may comprise a translucent material.

In a further embodiment, the channels 74, 76 may comprise a filter that only permits light to pass that has a certain wavelength corresponding to one or more desired wavelengths of the emitter 52. The filter would preferably reduce interference from other operating lights in a caregiver facility, other wavelengths of the emitters 52, or the like. In yet another embodiment, the application portion 66 or guide 64 may be transparent or translucent and/or may operate as the foregoing filter itself.

While the application portion 66 in FIG. 2 is shown to be substantially horizontal and the gripping portion is shown to be substantially vertical, it should be appreciated that other arrangements may also be used. Additionally, it should be appreciated that the guide 64 may comprise only one of either the application portion 66 or gripping portion 68.

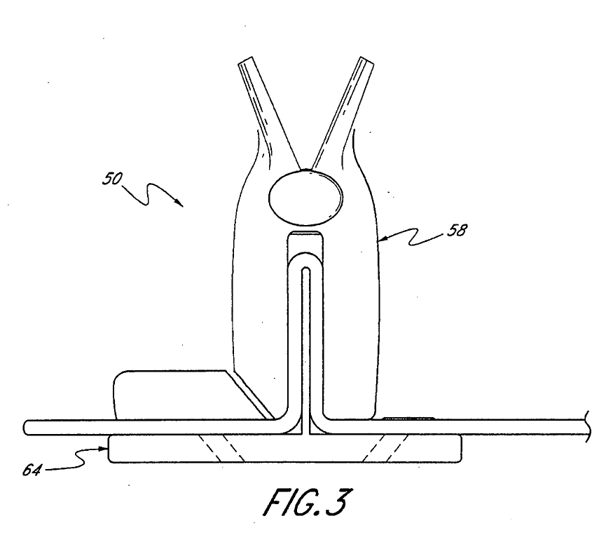

FIG. 3 illustrates a further embodiment of the positioning mechanisms disclosed above. In this embodiment, the sensor 50 preferably passes over the guide 64 as discussed with reference to FIG. 2, and the clip 58 is placed so as to secure the sensor 50 over the guide 64 and to facilitate gripping and application by the caregiver.

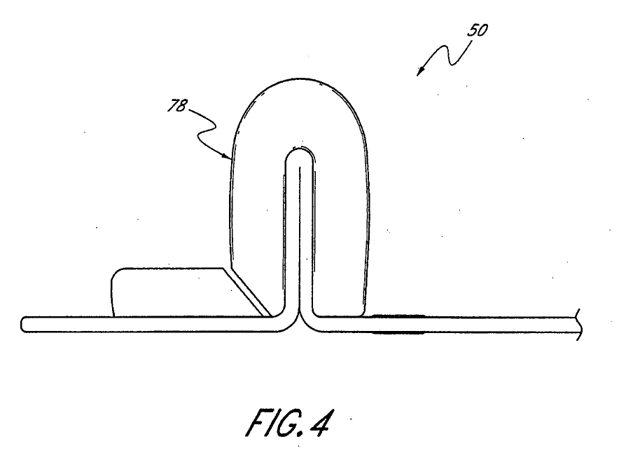

FIG. 4 illustrates yet another embodiment of the sensor 50. In this embodiment, a fitted clamp 78 is placed over the folded portion 56 of the sensor 50 to secure the sensor 50 in place. Although it is not shown, it should be appreciated that the fitted clamp 78 may also be used with the guide 64.

Preferably, the clamp 78 is friction fitted to the sensor 50 and may be removed following identification of the patient. An artisan will recognize many ways to friction fit the clamp 78 to the sensor 50. For example, the clamp 78 may comprise a corrugated portion or a material that will increase the friction between the clamp 78 and the sensor 50. In a further embodiment, the clamp 78 may be snap fit to the sensor 50. One of ordinary skill in the art will recognize even further ways of attaching the clamp 78 to the sensor 50.

An artisan will recognize that various shapes of the clamp 78 will function to achieve the same purpose as the embodiment illustrated in FIG. 4. For example, in one embodiment, the clamp 78 may comprise tabs on one end to facilitate gripping the clamp 78. In another exemplary embodiment, the clamp 78 may comprise a corrugated gripping portion to also facilitate gripping.

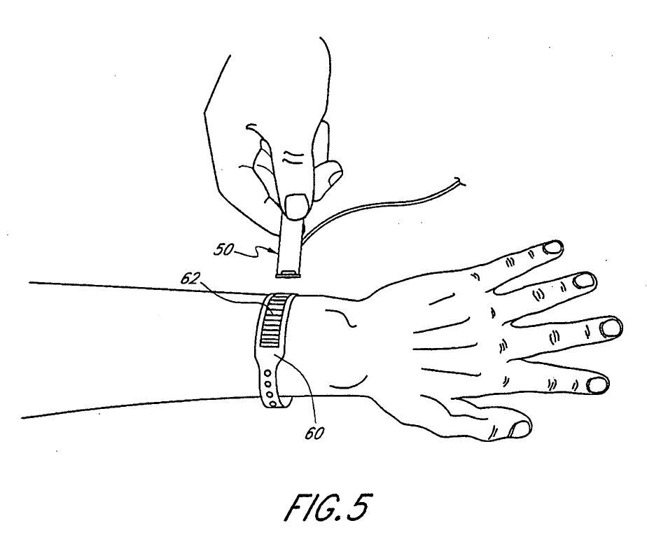

As shown in FIG. 5, a caregiver may identify a patient by passing the sensor 50 positioned using one or more of the positioning mechanisms of FIGS. 1A, 2, 3, and 4, over the bar code 62 on the identification bracelet 60 of the patient.

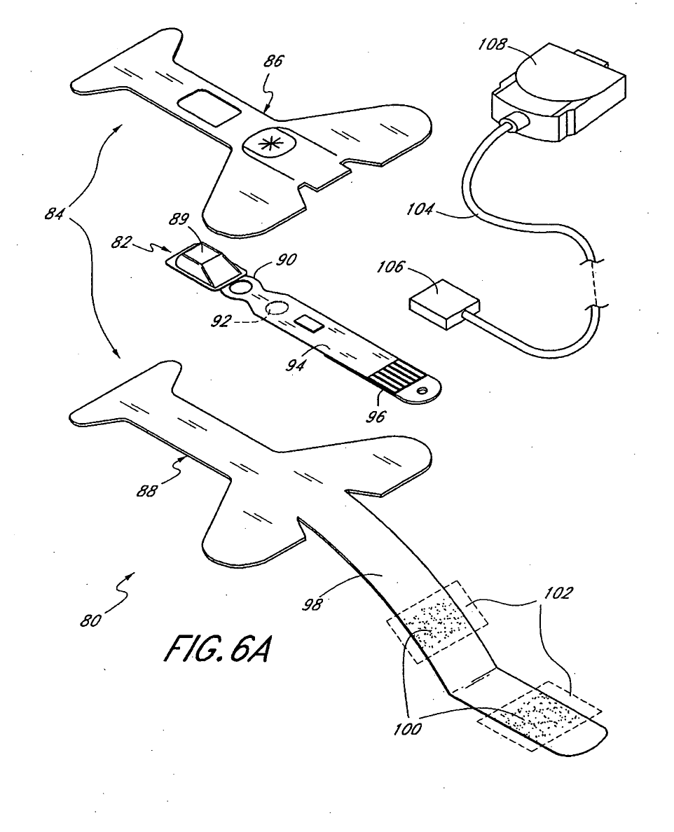

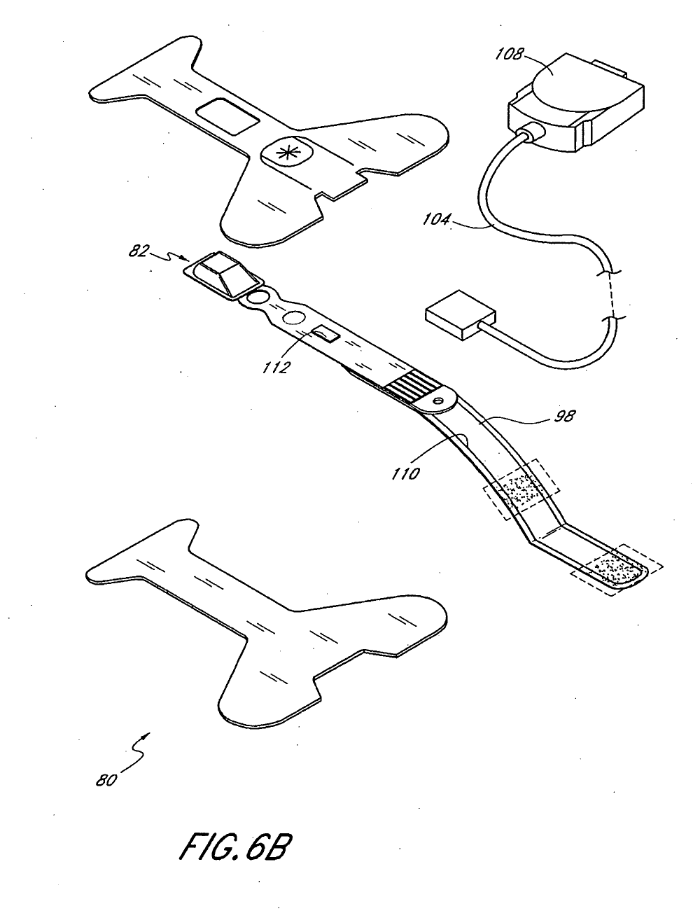

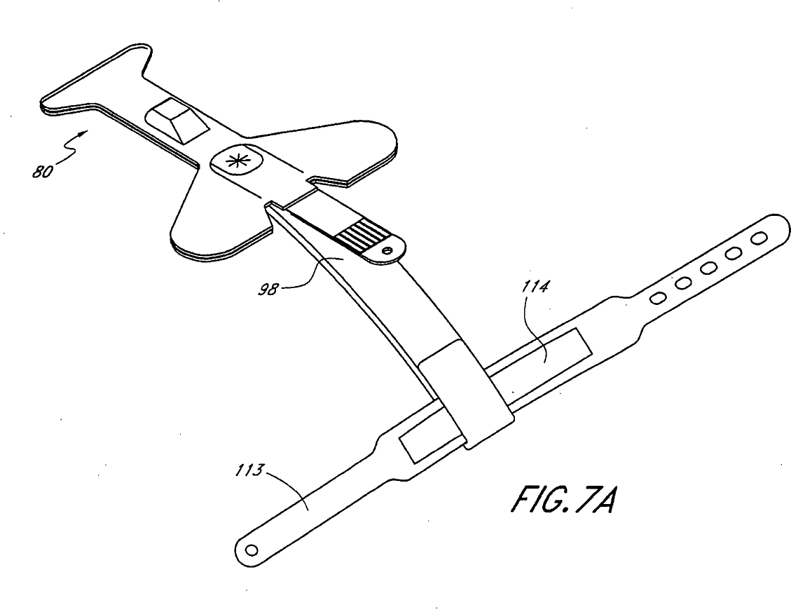

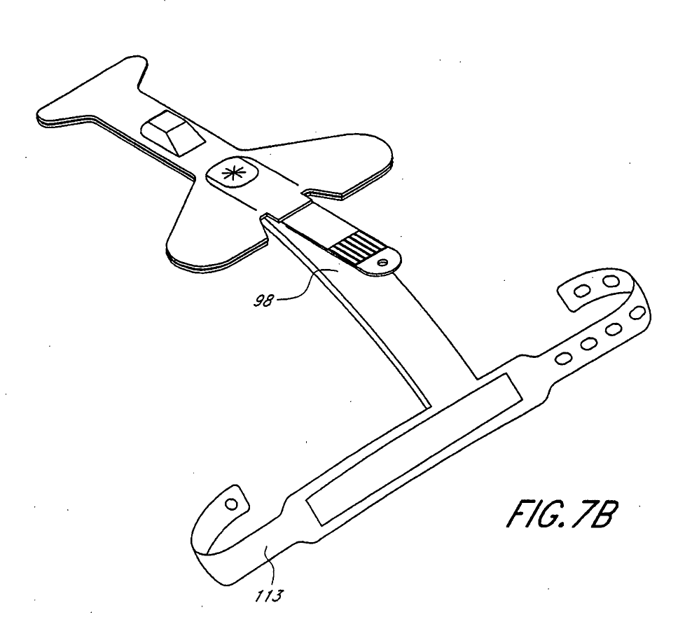

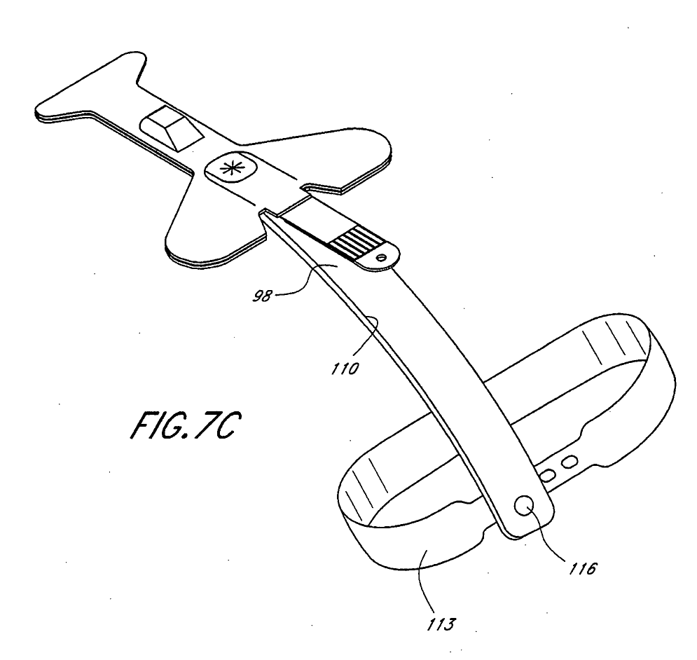

In yet another embodiment, it may be convenient or practical to interconnect the sensor 50 to an identification bracelet. In this embodiment, the identification bracelet may or may not have bar codes to identify the patient. FIGS. 6A through 7C illustrate various embodiments of attachment mechanisms. FIG. 6A illustrates an exemplary embodiment of a disposable sensor 80. The disposable sensor 80 preferably comprises a reusable portion 82 and a disposable portion 84. In one embodiment, the disposable portion 84 comprises a face tape layer 86 and a base tape layer 88. Preferably, the reusable portion 82 comprises a photodetector 89, a light-piping barrier 90, an emitter 92, a flex circuit 94, and an electrical connector 96. The light-piping barrier 90 reduces interference with the emitted light during the sensor's use. The flex circuit 94 preferably extends from the photodetector 89 and the emitter 92 to the electrical connector 96.

The disposable sensor 80 is connected to an oximeter via a connection cable 104. A sensor connector 106 located on the one end of the connection cable 104 is configured to accommodate the electrical connector 96 of the reusable portion 82. On the other end of the connection cable 104 is an oximeter connector 108 sized and configured to interconnect with the oximeter. Preferably, the flex circuit 94 is sufficiently elongated so as to provide flexibility when the electrical connector 96 is connected to the connection cable 104. In application, the reusable portion 82 is preferably located between the face tape layer 86 and the base tape layer 88.

In one embodiment, the base tape layer 88 preferably comprises a securing portion 98 that is configured to be interconnected with, for example, the patient's identification bracelet. In the illustrated embodiment, the securing portion 98 is comprised of a strap that extends from a portion of the base tape layer 88. The securing portion 98 is preferably a sufficient length to accommodate connection with a patient's identification bracelet. As illustrated, the securing portion 98 may comprise an adhesive substrate 100 that is covered with a release liner 102 until application. In this embodiment, when applied, the release liner 102 is removed, exposing the adhesive substrate 100. The securing portion 98 is folded over the identification bracelet and attached to a corresponding portion of the securing portion 98.

While the illustrated embodiment shows the securing portion 98 substantially comprising a strap, it will be appreciated by an artisan from the disclosure herein that other ways may be provided for attaching the sensor 50 to the identification bracelet. For example, the sensor may be attached to the bracelet via a cord, a wire, or other securing means. Additionally, in these further embodiments, adhesive substrate may be used or other means of attaching the securing portion to the identification bracelet may be used, such as, for example, hook-and-loop material such as velcro®, snaps, rivets, or the like.

In one embodiment, the base tape layer 88 may be made of a material that permits light to pass of a certain wavelength that corresponds to light from the emitter 92. In this embodiment, the base tape layer 88 would operate as a filter to prevent other operating lights in a caregiver facility from reaching the photodetector 88.

FIG. 6B illustrates another embodiment of attaching the disposable sensor 80 to the identification bracelet of a patient. In this embodiment, the securing portion 98 is interconnected to the reusable portion 82 of the disposable sensor 80. This embodiment would permit continuous use of the reusable sensor elements without removing them from the patient's identification bracelet. In this embodiment, the securing portion 98 may be attached to the identification bracelet as described above with reference to FIG. 6A. Also illustrated in this embodiment is a security wire 110 and a patient information memory device 112. In one embodiment, patient information may be downloaded onto the patient information memory device 112, and the information may be retrieved by an oximeter system or other healthcare device via the connection cable 104. The security wire 110 and the patient information memory device 112 may be configured to form a circuit such that disconnection of the security wire 110 will remove patient information from the patient information memory device 112.

FIG. 7A illustrates attachment of the disposable sensor 80 of FIGS. 6A or 6B to the identification bracelet 113. While FIG. 7A illustrates the securing portion 98 enveloping the identification bracelet 113, it should also be appreciated that a flap may be provided on the back of the identification bracelet 113 such that the securing portion passes through the flap without interfering with the patient identification window 114.

In another embodiment, as shown in FIG. 1B, the securing portion 98 may be manufactured such that it is integrally formed with the patient identification bracelet 113. Although not shown, a security wire 110 may pass through the securing portion 98 and the bracelet 113 such that removal of either will erase the patient information from the memory device 112.

In yet a further embodiment, as shown in FIG. 7C, the securing portion 98 may be sized and configured to accommodate a clasp or rivet 116 that is used to secure the identification bracelet 113. As shown in this embodiment, the security wire 110 may encompass the clasp 116, such that removal of the securing portion 98 will sever the security wire 110 and erase the patient's specific information on the memory device 112. In another embodiment, severance of the security wire 110 will make the sensor inoperable.

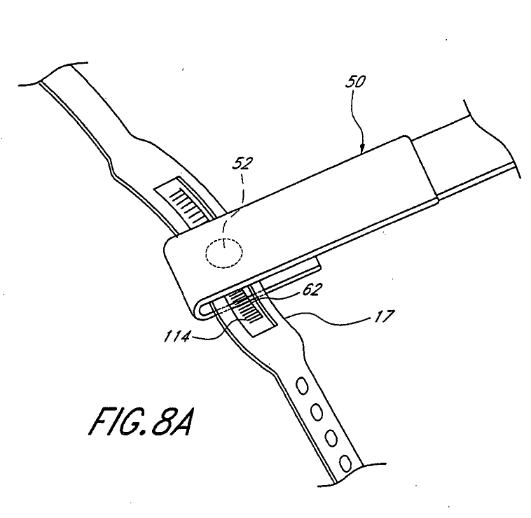

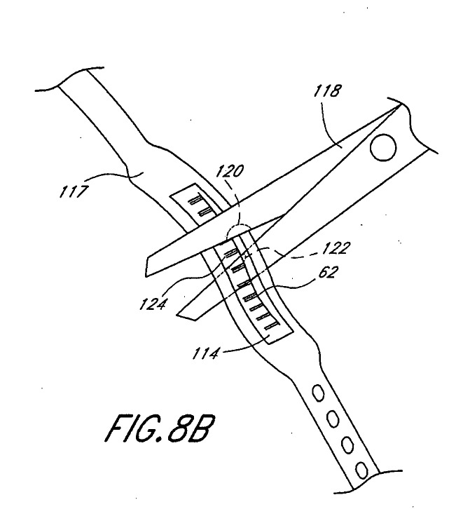

FIG. 8A illustrates another embodiment of using a physiological sensor 50 to identify a patient by reading the bar code 62 on the patient identification bracelet 117. In this embodiment, the sensor is folded over at a location between the emitter 52 and the photodetector (not shown) such that the emitter and the photodetector face each other. The patient identification bracelet 117 preferably includes a transparent window 114 such that light emitted from the emitter 52 will pass through the window 114 for detection by the photodetector. The identification bracelet is inserted between the emitter 52 and the photodetector and is advanced at a constant rate. In one embodiment, the window may comprise a material such that other operating lights in the caregiver facility are filtered out, thus reducing interference.

In another embodiment, shown in FIG. 8B, a reusable oximetry sensor 118 may be used. In this embodiment, the patient identification bracelet 117 with a transparent window 114 is placed between the emitter 120 and the photodetector 122. Emitted light 124 passes from the emitter 120 to the photodetector 122 through the transparent window 114. The identification bracelet is placed between the emitter 120 and photodetector 122 and is advanced at a constant rate such that the sensor 118 or the device to which the sensor 118 is connected will identify the bar code 62 pattern corresponding to the patient. An advantage of this embodiment is that the reusable sensor 118 requires no modification to the existing light paths.

Although the foregoing invention has been described in terms of certain preferred embodiments, other embodiments will be apparent to those of ordinary skill in the art. For example, some or all of the embodiments disclosed with reference to FIGS. 1A, 2, 4, 6A, 6B, 7A through 7C, 8A, and 8B, may be combined. Additionally, other combinations, omissions, substitutions, and modifications will be apparent to one of ordinary skill in the art in view of the disclosure herein. Accordingly, the present invention is not intended to be limited by the preferred embodiments, but is to be defined by reference to the appended claims.

What is claimed is:

1. A physiological sensor system usable to acquire information related to the wearer of a physiological sensor, the physiological sensor system comprising:
a physiological sensor adapted to be attached to a patient and including at least one emitter and a photodetector; and
a positioning element that positions that physiological sensor such that the at least one emitter is sufficiently proximate the detector to acquire information from an identification element worn by the patient.

2. The sensor system of claim 1, wherein the positioning device is a clip.

3. The sensor system of claim 1, wherein the positioning device is a guide.

4. The sensor system of claim 3, wherein the guide comprises a filter.

5. The sensor system of claim 3, wherein the guide comprises at least one channel.

6. The sensor system of claim 5, wherein the at least one channel further comprises a transparent material.

7. The sensor system of claim 3, wherein the guide is transparent.

8. The sensor system of claim 1, wherein the positioning device is a clamp.

9. The sensor system of claim 1, wherein the positioning device is a guide and one of a clip and a clamp.

10. The sensor system of claim 9 further comprising a filter.

11. The sensor system of claim 1, wherein the identification element is a wristband.

12. The sensor system of claim 11, wherein the identification element comprises a bar code.

13. A method of using a physiological sensor system to acquire information related to the wearer of a physiological sensor, the method comprising the steps of:
providing a physiological sensor including at least one emitter and a photodetector;
providing a positioning element that positions the physiological sensor such that the at least one emitter is sufficiently proximate the detector to acquire information from an identification element on the patient; and
acquiring information from an identification element on the patient via the physiological sensor.

14. The method of claim 13, wherein the information is acquired from an identification element via a pulse oximetry sensor.

15. The method of claim 13, wherein the positioning element comprises a guide.

16. The method of claim 13, wherein the positioning element comprises a clip.

17. The method of claim 13, wherein the positioning element comprises a clamp.

18. The method of claim 13, wherein the positioning element comprises a guide and one of a clip and a clamp.
